(19) United States
(12) Patent Application Publication
(10) Pub. No.: US 2024/0279156 A1
(43) Pub. Date: Aug. 22, 2024

(54) Novel diamine compound, reactive monomer for polyimide precursor containing the same, and polyimide film prepared therefrom

(71) Applicant: PUSAN NATIONAL UNIVERSITY INDUSTRY-UNIVERSITY COOPERATION FOUNDATION, Busan (KR)

(72) Inventors: Chang Sik HA, Busan (KR); Jun Seok LEE, Busan (KR); Sung Soo PARK, Busan (KR); Jadhav Amol MARUTI, Busan (KR)

(73) Assignee: PUSAN NATIONAL UNIVERSITY INDUSTRY-UNIVERSITY COOPERATION FOUNDATION, Busan (KR)

(21) Appl. No.: 18/413,582

(22) Filed: Jan. 16, 2024

(30) Foreign Application Priority Data
Jan. 16, 2023 (KR) 10-2023-0006068

(51) Int. Cl. C07C 217/84; C07C 201/12; C07C 213/02; C08G 73/10

(52) U.S. Cl. CPC C07C 217/84; C07C 201/12; C07C 213/02; C08G 73/105

(57) ABSTRACT

Disclosed are a novel diamine compound, a reactive monomer for a polyimide precursor containing the same, and a polyimide film manufactured therefrom. The novel diamine compound is represented by a following Chemical Formula 1:

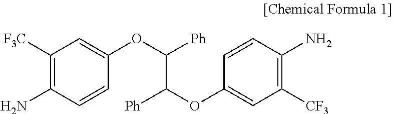 [SMILES: Nc1ccc(OC(c2ccccc2)C(Oc2ccc(N)c(C(F)(F)F)c2)c2ccccc2)cc1C(F)(F)F]

where in the Chemical Formula 1, Ph represents a substituted or unsubstituted phenyl group.

### BACKGROUND

### Field

The present disclosure relates to a novel diamine compound, a reactive monomer for a polyimide precursor containing the same, and a polyimide film manufactured therefrom.

### Description of Related Art

Polyimide is known as a material that may be applied to industries such as next-generation wireless communications and electronic devices because it has excellent chemical resistance and appropriate dielectric properties. Recently, polyimide is mainly used as a substrate material for flexible displays.

However, polyimide, in particular fluorinated polyimide, is often damaged due to the high temperature conditions in spite of its good optical transparency when applied during the manufacturing of flexible displays, resulting in product defects. Moreover, conventional polyimide including fluorinated polyimide has a problem that dielectric properties thereof in a wide range of high frequencies are still high. Therefore, research is needed to prepare polyimide with transparency, high heat resistance, as well as desirable dielectric constant and dielectric loss rate in a wide range of high frequencies.

### SUMMARY

A purpose of the present disclosure is to provide a novel diamine compound and a reactive monomer for a polyimide precursor containing the same.

Another purpose of the present disclosure is to provide a polyimide precursor prepared from the reactive monomer for the polyimide precursor, and a fluorinated polyimide film having high heat resistance manufactured using the same.

Purposes according to the present disclosure are not limited to the above-mentioned purpose. Other purposes and advantages according to the present disclosure that are not mentioned may be understood based on following descriptions, and may be more clearly understood based on embodiments according to the present disclosure. Further, it will be easily understood that the purposes and advantages according to the present disclosure may be realized using means shown in the claims and combinations thereof.

A first aspect of the present disclosure provides a diamine compound represented by a following Chemical Formula 1:

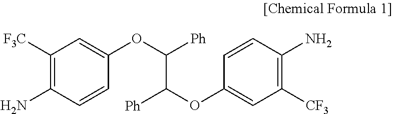 [SMILES: Nc1ccc(OC(c2ccccc2)C(Oc2ccc(N)c(C(F)(F)F)c2)c2ccccc2)cc1C(F)(F)F]

where in the Chemical Formula 1, Ph represents a phenyl group, or a phenyl group having a substituting alkyl group having 1 to 5 carbon atoms or a substituting trifluoromethyl group.

A second aspect of the present disclosure provides a method for preparing a diamine compound, the method comprising: reacting a compound represented by a following Chemical Formula 2 with a compound represented by a following Chemical Formula 3 under presence of a sodium hydride catalyst to synthesize a dinitro compound represented by a following Chemical Formula 4; and hydrogenating the dinitro compound under presence of a palladium/carbon catalyst:

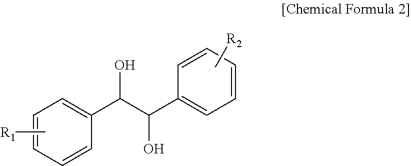

where in the Chemical Formula 2, each of R₁ and R₂ independently represents hydrogen, an alkyl group having 1 to 5 carbon atoms, or a trifluoromethyl group,

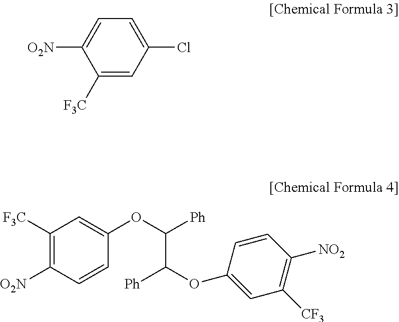 [SMILES: O=[N+]([O-])c1ccc(Cl)cc1C(F)(F)F ; O=[N+]([O-])c1ccc(OC(c2ccccc2)C(Oc2ccc([N+](=O)[O-])c(C(F)(F)F)c2)c2ccccc2)cc1C(F)(F)F]

where in the Chemical Formula 4, Ph represents a phenyl group, or a phenyl group having a substituting alkyl group having 1 to 5 carbon atoms or a substituting trifluoromethyl group.

In one embodiment of the method, the reaction for synthesizing the dinitro compound is carried out in an organic solvent at a temperature of 110 to 140° C.

A third aspect of the present disclosure provides a reactive monomer for a polyimide precursor, the reactive monomer containing the diamine compound as described above.

A fourth aspect of the present disclosure provides a polyimide precursor prepared by polymerizing the reactive monomer for the polyimide precursor as described above and a polymerization component including at least one acid dianhydride with each other.

In one embodiment of the polyimide precursor, the acid dianhydride is selected from a group consisting of pyromellitic dianhydride, 3,3,4,4-biphenyltetracarboxylic dianhydride, 4,4′-(hexafluoroisopropylidene) diphthalic anhydride, and cyclobutane-1,2,3,4-tetracarboxylic dianhydride.

A fifth aspect of the present disclosure provides a polyimide film manufactured using a solution containing the polyimide precursor as described above.

In one embodiment of the polyimide film, a glass transition temperature of the polyimide film is 215° C. or higher.

The novel diamine compound according to the present disclosure includes a trifluoromethyl aniline group, an ether group, and a phenyl group. Since the diamine compound of the present disclosure with this structure includes a thermally stable ether group, the polyimide synthesized using the same may exhibit high thermal stability.

In addition, the novel diamine compound in accordance with the present disclosure contains bulky phenyl and trifluoromethyl groups. Thus, when the polyimide polymer has been synthesized using the same, charge transfer complex unique to the polyimide polymer may be suppressed, and a polyimide film that is transparent and has low dielectric constant and dielectric loss may be manufactured. Therefore, the polyimide prepared from the novel diamine compound in accordance with the present disclosure may be applied to 5G and 6G wireless communications.

In addition to the effects as described above, specific effects in accordance with the present disclosure will be described together with following detailed descriptions for carrying out the disclosure.

### BRIEF DESCRIPTION OF DRAWINGS

FIG. 1 is a schematic diagram showing a synthesis process of a novel diamine compound according to one embodiment of the present disclosure.

FIGS. 2 to 4 show hydrogen nuclear magnetic resonance spectra and carbon nuclear magnetic resonance spectra of materials synthesized according to an embodiment of the present disclosure, respectively.

FIGS. 5 to 6 respectively show FT-IR spectra of materials synthesized according to one embodiment of the present disclosure.

FIG. 7 shows a FT-IR spectrum of polyimide prepared using the novel diamine compound in accordance with the present disclosure.

FIG. 8 is a Differential Scanning Calorimeter (DSC) graph measuring a glass transition temperature of each of polyimide according to Present Example of the present disclosure, and polyimide (6-FDA-DPEDBA) according to Comparative Example.

FIG. 9 is a thermogravimetric analysis (TGA) graph measuring thermal stability of each of polyimide according to Present Example of the present disclosure, and polyimide (6-FDA-DPEDBA) according to Comparative Example.

### DETAILED DESCRIPTIONS

Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims. Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, and components have not been described in detail so as not to unnecessarily obscure aspects of the present disclosure.

The terminology used herein is for the purpose of describing particular embodiments only and is not intended to limit the present disclosure. As used herein, the singular forms “a” and “an” are intended to include the plural forms as well, unless the context clearly indicates otherwise. It will be further understood that the terms “comprise”, “comprising”, “include”, and “including” when used in this specification, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of the associated listed items. Expression such as “at least one of” when preceding a list of elements may modify an entirety of list of elements and may not modify the individual elements of the list. When referring to “C to D”, this means C inclusive to D inclusive unless otherwise specified.

It will be understood that, although the terms “first”, “second”, “third”, and so on may be used herein to illustrate various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

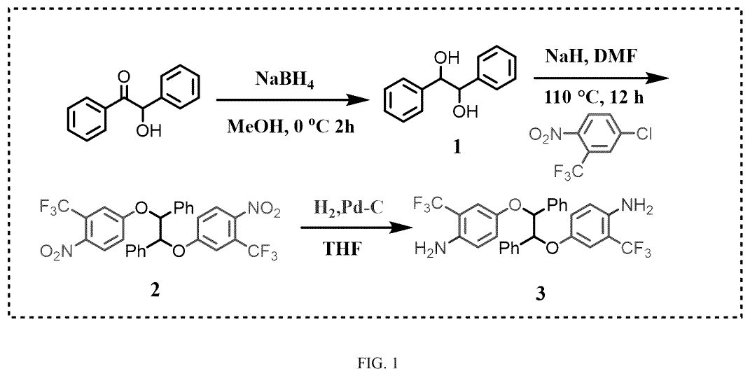

FIG. 1 is a schematic diagram showing the synthesis process of a novel diamine compound according to one embodiment of the present disclosure.

Referring to FIG. 1, the diamine compound according to one embodiment of the present disclosure may be represented by a following Chemical Formula 1:

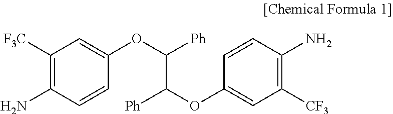 [SMILES: Nc1ccc(OC(c2ccccc2)C(Oc2ccc(N)c(C(F)(F)F)c2)c2ccccc2)cc1C(F)(F)F]

In the Chemical Formula 1, Ph represents a substituted or unsubstituted phenyl group. More specifically, Ph may represent a phenyl group, or a phenyl group having a substituting alkyl group having 1 to 5 carbon atoms or a substituting trifluoromethyl group.

The novel diamine compound in accordance with the present disclosure as represented by Chemical Formula 1 includes a trifluoromethyl aniline group, an ether group, and a phenyl group. The diamine compound of the present disclosure which has the above structure includes a thermally stable ether group. Thus, the polyimide synthesized using the same may exhibit high thermal stability.

In addition, the novel diamine compound in accordance with the present disclosure contains bulky phenyl and trifluoromethyl groups. Thus, when the polyimide polymer has been synthesized using the same, charge transfer complex unique to the polyimide polymer may be suppressed, and a polyimide film that is transparent and has low dielectric constant and dielectric loss may be manufactured. Therefore, the polyimide prepared from the novel diamine compound in accordance with the present disclosure may be applied to 5G and 6G wireless communications.

Further, as shown in FIG. 1, the novel diamine compound according to one embodiment of the present disclosure may be prepared by a method including reacting a compound represented by a following Chemical Formula 2 with a compound represented by a following Chemical Formula 3 under presence of a sodium hydride catalyst to synthesize a dinitro compound represented by a following Chemical Formula 4; and hydrogenating the dinitro compound under presence of a palladium/carbon catalyst:

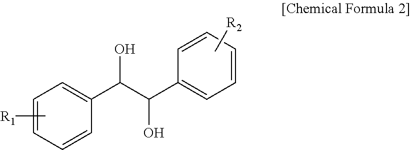

where in the Chemical Formula 2, each of R₁ and R₂ independently represents hydrogen, an alkyl group having 1 to 5 carbon atoms, or a trifluoromethyl group,

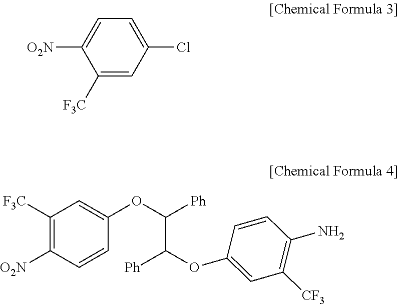 [SMILES: O=[N+]([O-])c1ccc(Cl)cc1C(F)(F)F ; Nc1ccc(OC(c2ccccc2)C(Oc2ccc([N+](=O)[O-])c(C(F)(F)F)c2)c2ccccc2)cc1C(F)(F)F]

where in the Chemical Formula 4, Ph represents a substituted or unsubstituted phenyl group. More specifically, Ph may represent a phenyl group, or a phenyl group having a substituting alkyl group having 1 to 5 carbon atoms or a substituting trifluoromethyl group.

In one embodiment, the reaction for synthesizing the dinitro compound may be performed in an organic solvent at a temperature of 110 to 140° C. For example, the organic solvent may include N,N-dimethylmethanamide, N,N-dimethylacetamide, dimethyl sulfoxide, etc. However, an embodiment is not limited thereto.

More specifically, the novel diamine compound in accordance with the present disclosure as represented by the Chemical Formula 1 may be prepared by synthesizing the dinitro compound via a condensation reaction of the compound represented by the Chemical Formula 2 and the compound represented by the Chemical Formula 3 using sodium hydride as a catalyst, and performing a hydrogenation reaction on the dinitro compound using palladium/carbon as a catalyst.

Moreover, the reactive monomer for the polyimide precursor according to one embodiment of the present disclosure may contain the diamine compound.

Moreover, the reactive monomer for the polyimide precursor according to one embodiment of the present disclosure and the polymerization component including at least one acid dianhydride may be subjected to a condensation polymerization reaction to synthesize into the polyimide precursor. In this regard, the acid dianhydride may be selected from a group consisting of pyromellitic dianhydride, 3,3,4,4-biphenyltetracarboxylic dianhydride, 4,4′-(hexafluoroisopropylidene) diphthalic anhydride, and cyclobutane-1,2,3,4-tetracarboxylic dianhydride. However, an embodiment is not limited thereto. Any acid dianhydride may be employed as long as it may be polymerized with the diamine compound to synthesize the polyimide precursor.

In another embodiment of the present disclosure, a polyimide film may be manufactured using a solution containing the polyimide precursor. In one embodiment, the polyimide precursor may be dissolved in an organic solvent to prepare a solution. The solution may be applied on a substrate, then the solvent may be dried and removed therefrom, and a thermal imidization process may be performed thereon. Thus, the polyimide film may be synthesized. In this regard, the thermal imidization process may be performed, for example, at 80° C. for 12 hours, at 150° C. for 1 hour, at 200° C. for 1 hour, at 250° C. for 1 hour, and at 300° C. for 1 hour. However, an embodiment is not limited thereto. The thermal imidization process may be performed in a known manner.

The polyimide films according to the present disclosure may exhibit excellent performance.

In one embodiment, the polyimide film had a glass transition temperature of 215° C. or higher, and thus exhibits high heat resistance. In other words, the polyimide film manufactured using the novel diamine compound according to the present disclosure has high heat resistance and thus has high potential for application to industries such as next-generation wireless communications and electronic devices.

Hereinafter, specific Examples and Experimental Examples according to the present disclosure will be described in detail. However, the following Examples are only some embodiments of the present disclosure. The scope of the present disclosure is not limited to the following Examples.

### PRESENT EXAMPLE 1

### Synthesis of 1,2-Diphenylethane-1,2-Diol as Material 1

5.0 g of benzoin and 160 ml of methanol were added into a 500 ml round flask, and were stirred for 1 hour, and then 2.0 g of sodium borohydride was slowly added thereto, followed by stirring for 2 hours. After the stirring, 80 ml of distilled water was added thereto to stop the reaction, and then, dichloromethane was added thereto using a separatory funnel to separate an organic layer therefrom, and moisture was absorbed therefrom with sodium sulfate, and then a filtering process was preformed thereon. Afterwards, a resulting product was concentrated using a rotary concentrator to obtain a powder material which in turn was dried in an oven at 80° C. for 15 hours to obtain a final material, that is, 1,2-diphenylethane-1,2-diol as the material 1.

### PRESENT EXAMPLE 2

### Synthesis of 1,2-Bis(4-Nitro-3-(Trifluoromethyl)Phenoxy)-1,2-Diphenylethane as Material 2

5.0 mmol of 1,2-diphenylethane-1,2-diol, 5.0 mmol of 2-chloro-5-nitrobenzotrifluoride, and 50 ml of N, N-dimethylmethanamide were input into a 100 ml round flask, which in turn was sealed under a nitrogen atmosphere. Afterwards, 2 g of sodium hydride was divided into several portions which in turn were added to the solution in the flask under an environment of 0° C. The mixed solution was stirred at 0° C. for 20 minutes, stirred at 25° C. for 30 minutes, and then stirred at 110° C. for 12 hours. After the reaction has been completed, the solution was cooled down and added to a dichloromethene/hexane (1:1) solution to produce a white precipitate, which in turn was filtered and then recrystallized using ethyl acetate and hexane. The recrystallized final compound was dried in a vacuum oven at 80° C. for 15 hours to produce a final material, that is, 1,2-bis(4-nitro-3-(trifluoromethyl)phenoxy)-1,2-diphenylethane as the material 2.

### PRESENT EXAMPLE 3

### Synthesis of 4,4′-((1,2- Diphenylethane-1,2-Diyl)Bis(Oxy))Bis(2-(Trifluoromethyl)Aniline) as Material 3

10 ml of 1,2-bis(4-nitro-3-(trifluoromethyl)phenoxy)-1,2-diphenylethane as the material 2, palladium/carbon catalyst, and a tetrahydrofuran solution were input into a 50 ml round flask, followed by stirring for 15 minutes under a nitrogen atmosphere. Afterwards, the flask was repeatedly subjected to a filling and vacuuming process using a hydrogen gas balloon at least three times to generate a hydrogen environment within the flask. The solution was then stirred for 12 hours. The completion of the reaction was observed using thin layer chromatography. After the reaction has been completed, the catalyst was filtered out from the solution using Celite 545 and the solution was concentrated using a rotary evaporator. The concentrated product was subjected to recrystallization using ethanol and drying in a vacuum oven at 100° C. for 12 hours. Thus, a final compound, that is, 4,4′-((1,2-diphenylethane-1,2-diyl)bis(oxy))bis(2-(trifluoromethyl) aniline) as the material 3 was obtained.

### PRESENT EXAMPLE 4

### Preparation of Polyamic Acid and Polyimide

Polyamic acid as a precursor of polyimide was produced via the condensation polymerization reaction of the diamine compound as the material 3 synthesized according to Present Example 3 and dianhydride. In a 20 ml vial, 2.0 mmol of the novel diamine compound as the material 3 and 2.0 mmol of pyromellitic dianhydride were dissolved at a solid content of 25 wt % in dimethylacetamide solvent under a nitrogen atmosphere, followed by stirring at room temperature for 72 hours.

Thereafter, 1.5 mL of a polyamic acid solution was applied to a slide glass substrate, the solvent was dried and removed therefrom in an oven under a nitrogen atmosphere at 60° C. for 24 hours, and a thermal imidization process was performed thereon at 80° C. for 12 hours, at 150° C. for 1 hour, at 200° C. for 1 hour, at 250° C. for 1 hour, and at 300° C. for 1 hour under a nitrogen atmosphere. Thus, a final polyimide was obtained.

### Comparative Example

A polyimide (6-FDA-DPEDBA) film represented by a following Chemical Formula 5 was prepared as a Comparative Example.

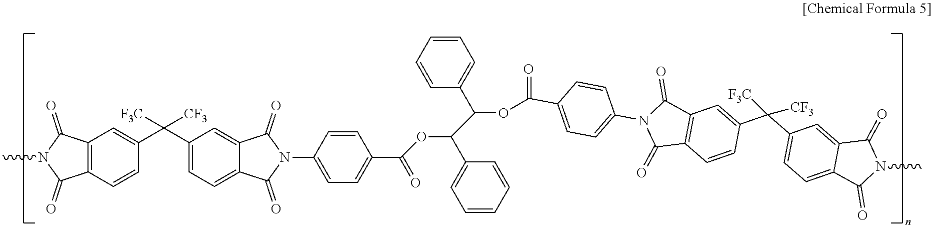

where, n is 8 to 75.

### Experimental Example 1

Chemical structures of 1,2-bis(4-nitro-3-(trifluoromethyl)phenoxy)-1,2-diphenylethane as the novel diamine precursor and the novel diamine compound in accordance with the present disclosure were identified through nuclear magnetic resonance analysis.

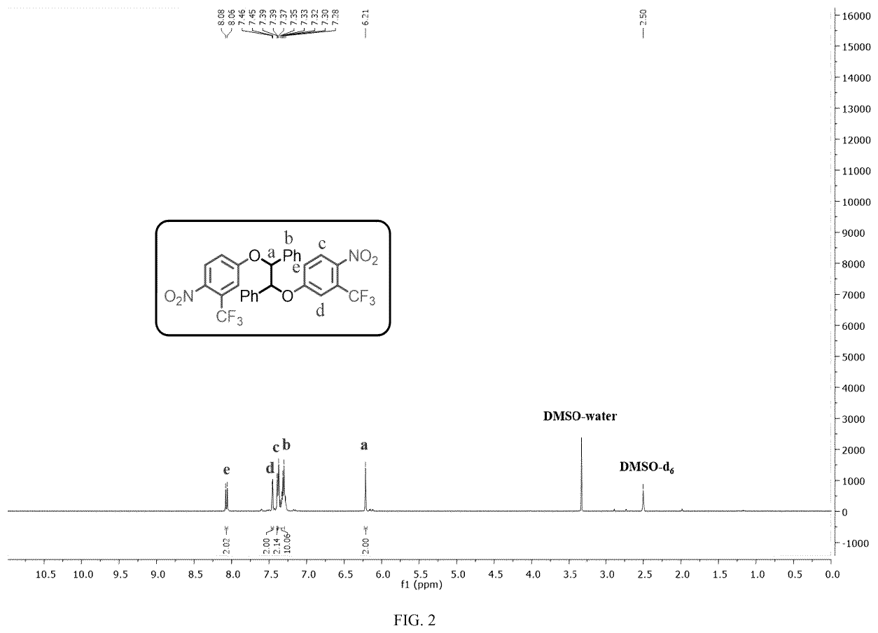

FIG. 2 is the hydrogen nuclear magnetic resonance spectrum of the dinitro compound. In FIG. 2, the peak appearing at 8.08 ppm is due to the hydrogen atom of the phenyl ring adjacent to the ether group, the peak appearing around 7.46 ppm is due to the hydrogen atom of the phenyl ring adjacent to the —CF₃ group, the peak appearing at 7.39 ppm is due to the hydrogen atom of the phenyl ring adjacent to the nitro group, and the peak appearing around 7.30 ppm is due to the hydrogen atoms of the side phenyl group, and the peak appearing at 6.21 ppm is due to the hydrogen atom of the ethyl group bonded to the side phenyl group.

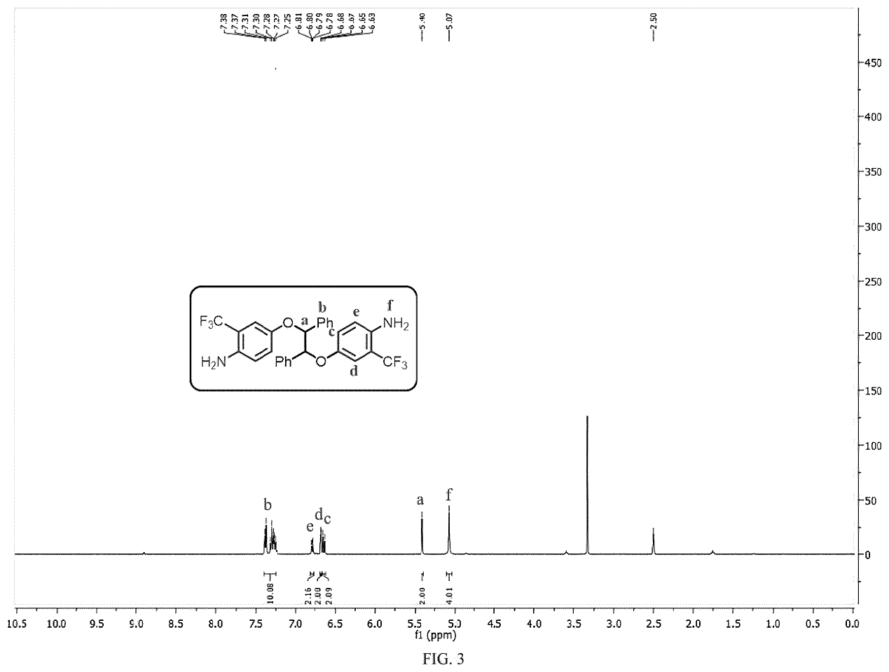

FIG. 3 shows the hydrogen nuclear magnetic resonance spectrum of the novel diamine compound according to one embodiment of the present disclosure. In FIG. 3, the peaks that appear around 7.37 ppm are due to the hydrogens of the side phenyl group, the peak appearing around 6.80 ppm is due to the hydrogen of the phenyl ring adjacent to the amine group, the peaks appearing at 6.78 and 6.65 ppm are due to the hydrogens of the phenyl ring adjacent to the ether group, the peak appearing at 5.40 ppm is due to the hydrogen of the ethyl group bonded to the side phenyl group, and the peak appearing at 5.07 ppm is due to the hydrogen of the amine group.

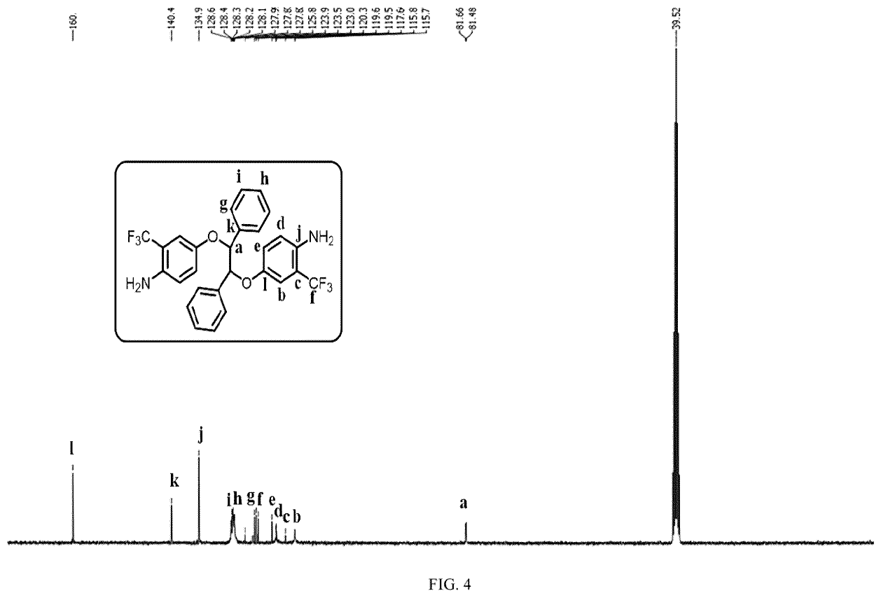

FIG. 4 shows the carbon nuclear magnetic resonance spectrum of the novel diamine compound according to one embodiment of the present disclosure. In FIG. 4, the peaks appearing at 115.7 to 160.1 ppm are due to the carbon atoms of the phenyl ring, and the peaks appearing at 160.1, 140.4, and 134.9 ppm appear at higher ppm due to the electronegativity of nitrogen and oxygen atoms. Moreover, the peak that appears at 81.48 ppm is due to the carbon of the ethyl group. Based on this result, it is identified that a target diphenylethane group is successfully introduced in the diamine compound.

### Experimental Example 2

Whether each of the novel diamine compound as prepared in the present disclosure, the polyimide and the precursor thereof prepared using the compound had been successfully synthesized was identified via functional group analysis.

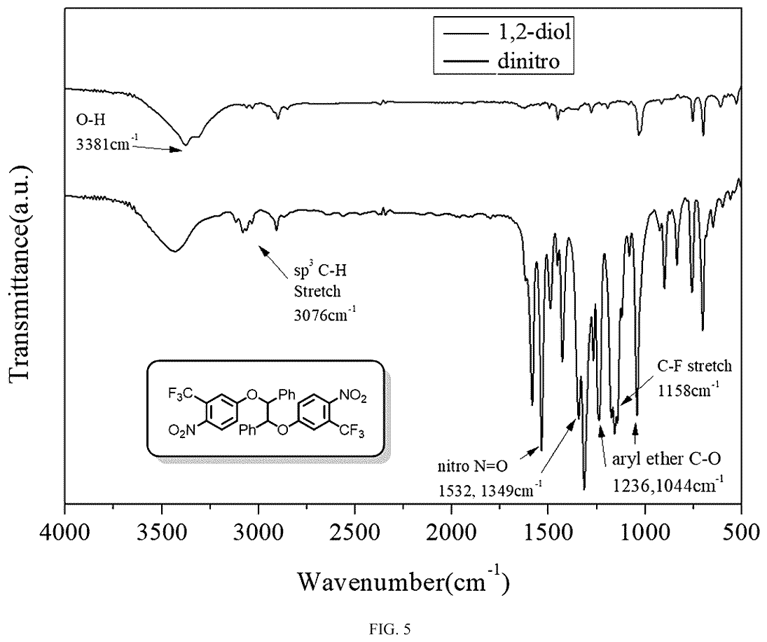

In the FT-IR spectrum of the 1,2-diphenylethane-1,2-diol compound (red line) as shown in FIG. 5, a peak appearing at 3381 cm⁻¹ is due to the alcohol group of the diol. Further, in the FT-IR spectrum of 1,2-bis(4-nitro-3-(trifluoromethyl)phenoxy)-1,2-diphenylethane compound (dinitro compound) (black line), peaks appear at 3076, 1532, 1349, 1236, 1158, and 1044 cm⁻¹. The peak appearing at 3076 cm⁻¹ is due to C-H of the diphenylethane group, and the peaks appearing at 1532 and 1349 cm⁻¹ are due to N═O of the nitro group. The peaks appearing at 1236 and 1044 cm⁻¹ are due to C—O of the ether group, and the peaks appearing at 1158 cm⁻¹ are due to C—F of the —CF₃ group.

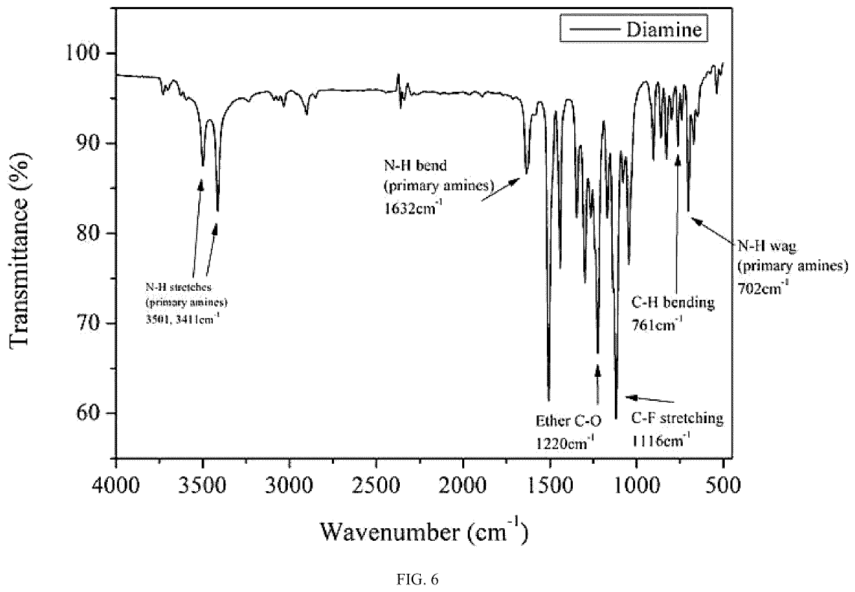

FIG. 6 shows the FT-IR spectrum of the novel diamine compound according to one embodiment of the present disclosure. In FIG. 6, peaks appear at 3501, 3411, 1632, 1220, 1116, 761, and 702 cm⁻¹. The peaks appearing at 3501 and 3411 cm⁻¹ are due to N—H stretching of the amine, and the peaks appearing at 1632 cm⁻¹ are due to N—H bending of the amine. The peak appearing at 1220 cm⁻¹ is due to C—O of the ether group, and the peak appearing at 1116 cm⁻¹ is due to C—F of the —CF3 group. The peaks appearing at 761 and 702 cm⁻¹ are due to C—H and N—H, respectively.

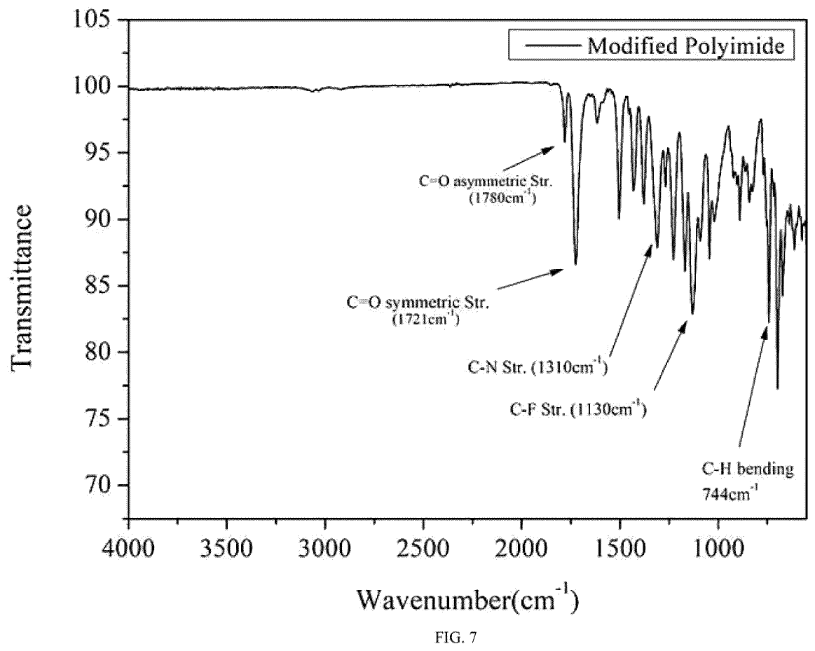

FIG. 7 shows the FT-IR spectrum of polyimide prepared in accordance with the present disclosure. In FIG. 7, peaks appear at 1780, 1721, 1310, 1130, and 744 cm⁻¹. The peak appearing at 1780 cm⁻¹ is due to C═O asymmetric stretching of the polyimide group, and the peak appearing at 1721 cm⁻¹ is due to C═O symmetric stretching of the polyimide group. The peak appearing at 1310 cm⁻¹ is due to C—N stretching of the polyimide group, and the peak appearing at 1130 cm⁻¹ is due to C—F stretching in the diamine monomer. Lastly, the peak that appears at 744 cm⁻¹ is due to C—H bending in the diamine monomer.

### Experimental Example 3

### Thermal Stability Test

Thermal properties were measured through differential scanning calorimetry (DSC) and thermogravimetric analysis (TGA).

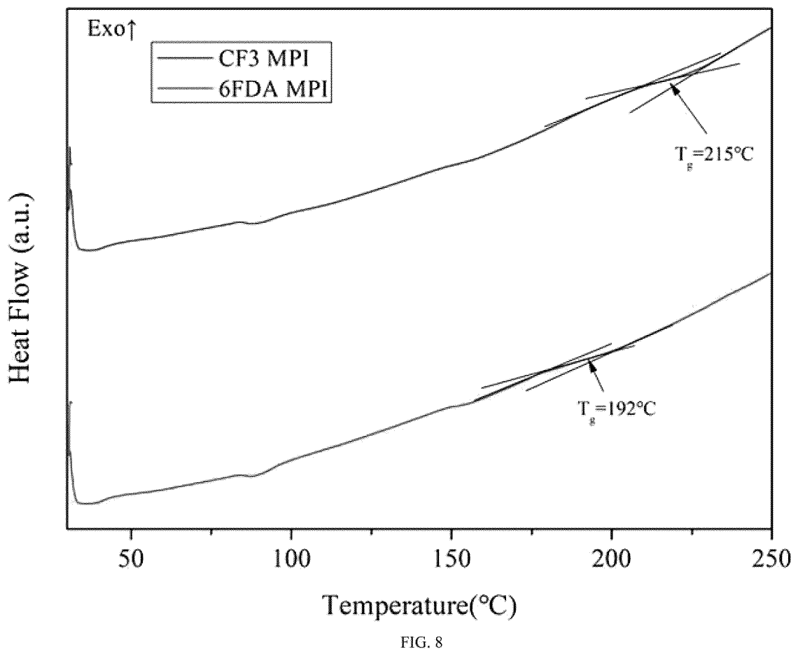

FIG. 8 is a graph showing the differential scanning calorimetry (DSC) result of each of polyimide (CF3 MPI) as prepared using the diamine compound as synthesized in accordance with the present disclosure and polyimide (6FDA MPI) prepared using 6FDA-DPEDBA (Comparative Example).

Referring to FIG. 8, the polyimide (6FDA MPI) prepared as Comparative Example has the glass transition temperature of 192° C., whereas the polyimide (CF3 MPI) prepared using the novel diamine compound according to Present Example of the present disclosure has a glass transition temperature of 215° C. Thus, it may be identified that the polyimide according to Present Example of the present disclosure has a higher glass transition temperature than that of the polyimide of Comparative Example.

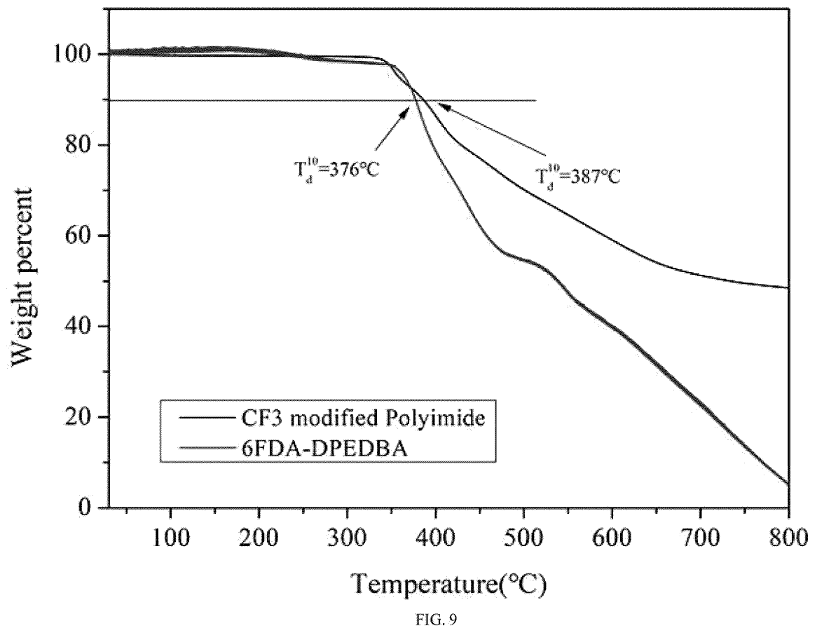

Referring to FIG. 9 showing the result of the thermogravimetric analysis (TGA), the 10 weight percent loss temperatures (Td¹⁰) of the polyimide (CF3 modified Polyimide) prepared according to Present Example and the polyimide (6FDA-DPEDBA) prepared according to Comparative Example are respectively 387° C. and 375° C. Thus, it may be identified that the polyimide film manufactured according to Present Example of the present disclosure has higher thermal stability than that of the polyimide film manufactured according to Comparative Example. This may be due to that in terms of the chemical structure, the polyimide prepared from the diamine compound containing the ether group instead of a thermally unstable ester group exhibits high thermal stability at high temperatures.

Although embodiments of the present disclosure have been described with reference to the accompanying drawings, embodiments of the present disclosure are not limited to the above embodiments, but may be implemented in various different forms. A person skilled in the art may appreciate that the present disclosure may be practiced in other concrete forms without changing the technical spirit or essential characteristics of the present disclosure. Therefore, it should be appreciated that the embodiments as described above is not restrictive but illustrative in all respects.

What is claimed is:

1. A diamine compound represented by a following Chemical Formula 1: 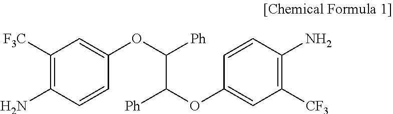 [SMILES: Nc1ccc(OC(c2ccccc2)C(Oc2ccc(N)c(C(F)(F)F)c2)c2ccccc2)cc1C(F)(F)F]
where in the Chemical Formula 1, Ph represents a phenyl group, or a phenyl group having a substituting alkyl group having 1 to 5 carbon atoms or a substituting trifluoromethyl group.

2. A method for preparing a diamine compound, the method comprising:
reacting a compound represented by a following Chemical Formula 2 with a compound represented by a following Chemical Formula 3 under presence of a sodium hydride catalyst to synthesize a dinitro compound represented by a following Chemical Formula 4; and
hydrogenating the dinitro compound under presence of a palladium/carbon catalyst: 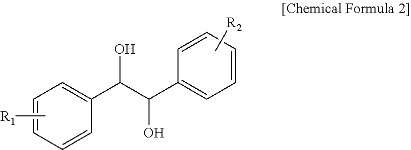
where in the Chemical Formula 2, each of R₁ and R₂ independently represents hydrogen, an alkyl group having 1 to 5 carbon atoms, or a trifluoromethyl group, 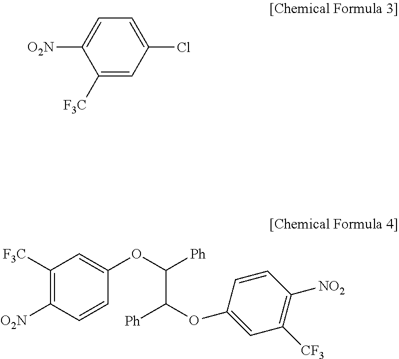 [SMILES: O=[N+]([O-])c1ccc(Cl)cc1C(F)(F)F ; O=[N+]([O-])c1ccc(OC(c2ccccc2)C(Oc2ccc([N+](=O)[O-])c(C(F)(F)F)c2)c2ccccc2)cc1C(F)(F)F]
where in the Chemical Formula 4, Ph represents a phenyl group, or a phenyl group having a substituting alkyl group having 1 to 5 carbon atoms or a substituting trifluoromethyl group.

3. The method of claim 2, wherein the reaction for synthesizing the dinitro compound is carried out in an organic solvent at a temperature of 110 to 140° C.

4. A reactive monomer for a polyimide precursor, the reactive monomer containing the diamine compound according to claim 1.

5. A polyimide precursor prepared by polymerizing the reactive monomer for the polyimide precursor according to claim 4 and a polymerization component including at least one acid dianhydride with each other.

6. The polyimide precursor of claim 5, wherein the acid dianhydride is selected from a group consisting of pyromellitic dianhydride, 3,3,4,4-biphenyltetracarboxylic dianhydride, 4,4′-(hexafluoroisopropylidene) diphthalic anhydride, and cyclobutane-1,2,3,4-tetracarboxylic dianhydride.

7. A polyimide film manufactured using a solution containing the polyimide precursor according to claim 5.

8. The polyimide film of claim 7, wherein a glass transition temperature of the polyimide film is 215° C. or higher.
